SEQUENCE LISTING (5 sequences)

SEQ ID NO: 1 - DNA, 108 bp, synthetic construct
  misc_feature 1..108: note = Artificial Sequence
  source 1..108: mol_type = other DNA
atgattaaaattgcgagccgcaaatttctgggcgtggaaaacgtgtatgatattggcgtgcgccgcgatcataacttttttattaaaaacggcctgattgcgagcaac

SEQ ID NO: 2 - protein, 36 aa, synthetic construct
  REGION 1..36: note = Artificial Sequence
MIKIASRKFLGVENVYDIGVRRDHNFFIKNGLIASN

SEQ ID NO: 3 - DNA, 438 bp, synthetic construct
  misc_feature 1..438: note = Artificial Sequence
  source 1..438: mol_type = other DNA
ccagccttgccagaggatggcggcagcggcgccttcccgccaggccacttcaaggacccaaagcgcctgtactgcaaaaacgggggcttcttcctgcgcatccacccagacggccgcgttgacggggtccgcgagaagagcgaccctcacatcaagctacaacttcaagcagaagagcgcggagttgtgtctatcaaaggagtgtgtgctaaccgttacctggctatgaaggaagatggacgcttactggcttctaaatgtgttacggatgagtgtttcttttttgaacgcttggaatctaataactacaatacttaccgctcacgcaaatacaccagttggtatgtggcactgaaacgcactgggcagtataaacttggatccaaaacaggacctgggcagaaagctatcctttttcttccaatgtctgctaagagc

SEQ ID NO: 4 - protein, 146 aa, synthetic construct
  REGION 1..146: note = Artificial Sequence
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - DNA, 717 bp, synthetic construct
  misc_feature 1..717: note = symposized
  source 1..717: mol_type = other DNA
gaattctaatacgactcactatagggagattgtgagcggataacaatttgtttaactttaagaaggagaatgcatcatcaccatcaccacatgattaaaattgcgagccgcaaatttctgggcgtggaaaacgtgtatgatattggcgtgcgccgcgatcataacttttttattaaaaacggcctgattgcgagcaacccagccttgccagaggatggcggcagcggcgccttcccgccaggccacttcaaggacccaaagcgcctgtactgcaaaaacgggggcttcttcctgcgcatccacccagacggccgcgttgacggggtccgcgagaagagcgaccctcacatcaagctacaacttcaagcagaagagcgcggagttgtgtctatcaaaggagtgtgtgctaaccgttacctggctatgaaggaagatggacgcttactggcttctaaatgtgttacggatgagtgtttcttttttgaacgcttggaatctaataactacaatacttaccgctcacgcaaatacaccagttggtatgtggcactgaaacgcactgggcagtataaacttggatccaaaacaggacctgggcagaaagctatcctttttcttccaatgtctgctaagagctaaagacccggggcttaattaattaagctagcataaccccttggggcctctaaacgggtcttgaggggttttttgtctaga

(19) United States
(12) Patent Application Publication
(10) Pub. No.: US 2023/0151374 A1
(43) Pub. Date: May 18, 2023

(54) PREPARATION OF HUMAN BASIC FIBROBLAST GROWTH FACTOR BY USING BACILLUS SUBTILIS AND ENDONUCLEASE

(71) Applicant: DREAMTEC INTELLECTUAL PROPERTY LIMITED, HONG KONG (HK)

(72) Inventors: Shu Kun Christopher CHUNG, Hong Kong (HK); Wai Yeung KWONG, Hong Kong (HK)

(21) Appl. No.: 17/821,658

(22) Filed: Aug. 23, 2022

Related U.S. Application Data
(63) Continuation of application No. PCT/CN2021/075070, filed on Feb. 3, 2021.

(30) Foreign Application Priority Data
Mar. 11, 2020 (CN) 202010164821.4

(51) Int. Cl. C12N 15/75; C12N 15/52; C07K 14/50; C12N 9/12

(52) U.S. Cl. CPC C12N 15/75; C07K 14/503; C12N 9/1252; C12N 15/52; C12Y 207/07007; C07K 2319/21; C07K 2319/92; C12N 2800/101

(57) ABSTRACT

The present invention relates to the preparation of human basic fibroblast growth factor by using Bacillus subtilis and endonuclease. Specifically, the present invention provides a nucleic acid construct, which comprises an insert, and the insert comprises, from the 5′ end to the 3′ end, a polynucleotide sequence that encodes a short peptide affinity tag, a trans-splicing intein derived from Anabaena and an exogenous polypeptide; and wherein the short peptide affinity tag serves as an N-terminal extein of the trans-splicing intein, and the exogenous polypeptide serves as a C-terminal extein of the trans-splicing intein. The present invention further provides an expression vector and a host cell that comprise the construct, and a method for producing and purifying foreign proteins. The expression system and method of the present invention can significantly improve the expression efficiency of biologically active exogenous proteins, reduce the generation of inclusion bodies, simplify purification steps, greatly reduce purification costs, and are especially suitable for large-scale cultivation.

### TECHNICAL FIELD

The invention relates to the biological field, and generally relates to a system and method for expressing exogenous polypeptide.

### BACKGROUND ART

Efficient and cost-effective expression of exogenous polypeptides, especially cytokines, in their native forms is increasingly important for the study of cell biology such as stem cells. Take human basic fibroblast growth factor (basic fibroblast growth factor, bFGF for short, also known as FGF2) as an example. bFGF, a member of the fibroblast growth factor family, has multiple therapeutic uses in neurodegenerative diseases, heart disease, and difficult-to-heal wound-like lesions [1-3]. Furthermore, bFGF plays an important role in tissue development by inducing proliferation of fibroblasts and stem cells [4-8], and it also plays an important role in a mass production of stem cells. However, the current high production cost and low yield of bFGF protein hinder its commercial application in the pharmaceutical industry [9-10]. For example, bFGF protein is unstable and easily degraded under stem cell culture conditions, and routine replacement of the fresh medium containing commercially available bFGF can greatly increase R&D costs. To promote the development of stem cell research, it is crucial to improve the upstream production benefits of recombinant human bFGF.

Escherichia coli ( E.coli ) is widely used as a bacterial host to express recombinant proteins without post-translational modifications. Escherichia coli is widely used in the field of biotechnology due to its advantages of fast growth rate, low cost and easy-to-use. However, E. coli is a gram-negative bacterium with LPS outer membrane, and therefore the purified recombinant protein is generally accompanied by a large amount of endotoxin. The endotoxins may lead to undesirable toxic effects when the relevant recombinant proteins are used to treat tissue culture samples or animal subjects. Endotoxins are difficult to isolate by downstream purification processes unless endotoxin-free water and endotoxin removal kit are used. The use of related kits thus increases the production cost of the target protein.

By contrast, Bacillus subtilis is a Gram-positive bacterium that is considered: “Generally Recognized as Safe” (GRAS) by the FDA because it does not contain endotoxins [11]. Bacillus subtilis is capable of stably expressing exogenous polypeptides, and has been engineered to express the secreted endogenous and exogenous proteins [12], the main reasons are: 1. Bacillus subtilis expresses and secretes a large number of proteases at the end of its logarithmic growth phase, which adversely affects the stable expression and yield of exogenous proteins; 2. The secretion of some exogenous proteins into the medium will affect the growth of the host bacteria, which also affects the high-efficiency expression of exogenous proteins; 3. Compared with E. coli, genetic engineering operations are more difficult. These factors are believed to limit the use of Bacillus subtilis as a host cell.

Inteins are protein elements that are capable of self-cleavage from host proteins and catalyzed flanking sequences (extein) linked by peptide bonds. Intein excision does not require post-translational processing by accessory enzymes or cofactors. The self-cleavage process is called “protein splicing”. The segment of the internal protein sequence is called “intein” and the segment of the external protein sequence is called “extein”, where the upstream extein is called “N-terminal extein” and the downstream extein is called “C-terminal extein”.

Protein intein not only enrich the content of post-translational processing of genetic information, but also have a wide range of applications in protein purification. Intein can be classified into two types according to the presence or absence of homing endonuclease domains within the intein. One is a fully functional intein (maxi-intein), which has protein splicing activity and a homing endonuclease (homing endonuclease) sequence; the other is a mini-intein (mini-intein), which only has protein splicing activity. According to their existing form, it is divided into whole intein and isolated intein. The two spliced regions of the former coexist on the same polypeptide fragment. The two spliced regions of the latter are present on different polypeptide fragments and are therefore called split inteins or fragmented inteins. Whole inteins undergo cis-splicing, while split/fragmented inteins undergo trans-splicing.

Inteins have been widely used in protein purification. So far, more than 400 inteins have been found in organisms. Inteins of various origins and structures have been used to construct protein expression and purification systems. The cleavage reaction rates and conditions of different inteins are different, and the purification efficiency is also very different. However, the factors affecting intein fragmentation are currently not well understood.

There is a need in the art for systems and methods that can cost-effectively express foreign polypeptides.

### DISCLOSURE OF THE INVENTION

In one embodiments, the present invention provides a nucleic acid construct, which comprises an insert, and the insert comprises, from the 5′ end to the 3′ end, a polynucleotide sequence that encodes a short peptide affinity tag, a trans-splicing intein derived from Anabaena sp and an exogenous polypeptide; and wherein the short peptide affinity tag serves as an N-terminal extein of the trans-splicing intein, and the exogenous polypeptide serves as a C-terminal extein of the trans-splicing intein.

In one aspect, the intein is an intein of Anabaena DNA polymerase III unit (Asp DnaE).

In one aspect, the intein comprises an amino acid sequence having at least 75% sequence identity to SEQ ID NO:2 or consists of it.

In one aspect, the exogenous polypeptide is a fibroblast growth factor (FGF), such as basic fibroblast growth factor (bFGF), especially human bFGF.

In one aspect, the short peptide affinity tag has a length of about 4-15 amino acids, for example, a 5-15 x His tag, especially a 6 x His tag.

In one aspect, the nucleic acid construct further comprises one or more of the following elements: a promoter, an operator, an enhancer, and a ribosome binding site.

In one aspect, the nucleic acid construct comprises, from the 5′ end to the 3′ end, a nucleotide sequence that encodes T7 promoter-lac operator-ribosome binding site (RBS)-6 x His tag-Asp DnaE intein-bFGF -T7 transcription terminator.

In one aspect, the nucleic acid construct further comprises a first cloning site upstream of the insert and a second cloning site downstream of the insert, wherein the first cloning site and the second cloning site allow the nucleic acid construct to be inserted into an expression vector.

In another embodiment, the present invention provides an expression vector comprising the nucleic acid construct of the present invention.

In another embodiment, the present invention provides a transformed Bacillus subtilis comprising the expression vector of the present invention.

In another embodiment, the present invention provides a method of producing an exogenous polypeptide comprising culturing a transformed Bacillus subtilis of the present invention under conditions that allow the expression of the exogenous polypeptide.

In one aspect, the method of producing an exogenous polypeptide further comprises isolating the cultured Bacillus subtilis and then lysing to obtain a cell lysate, followed by isolating the exogenous polypeptide from the cell lysate by sequential use of cation exchange chromatography and heparin-agarose (HA) Chromatography.

### BRIEF DESCRIPTION OF THE DRAWINGS

FIG. 1 shows the diagram of a plasmid construction vector (10.4 kb) expressing the H6-DnaE-bFGF insert expression cassette according to one embodiment of the present invention. ori = origin of replication of Bacillus subtilis; AmpR = ampicillin resistance gene; lacI = lacI gene; T7 RNAP = T7 ribonucleic acid polymerase gene; bFGF = bFGF gene; Asp DnaE = Asp DnaE intein; H6 = 6x His label; RBS = ribosome binding site. Arrows indicate the direction of gene expression.

FIG. 2 shows the results of a bFGF Western blotting assay in a Bacillus subtilis host cell lysate sample according to one embodiment of the present invention. Lanes 0 h, 2 h, 4 h, 6 h and 8 h: represent samples collected from the cultures at 0 h, 2 h, 4 h, 6 h and 8 h after induction, respectively, load 5 µl of cell lysate on per lane. Lane-ve: 5 µl of cell lysate from pECBS1 vector cultures at 8 hours after induction.

FIG. 3 shows a time course study of shake flask culture of Bacillus subtilis bFGF according to one embodiment of the present invention. Culture samples were obtained at different time points before and after IPTG induction. FIG. 3A: Western blot analysis results of bFGF present in cell lysate (CL) samples, where 5 µl of cell lysate was loaded per lane. FIG. 3B: cell viability and quantification of bFGF. (-•-) indicates detectable bFGF levels; CFU refers to colony forming units. The viable cell counts were determined on common agar plates and plates with kanamycin, and are represented by (-•- ) and (-■-), respectively. Transformant growth experiments were repeated in triplicate and standard error bars are shown.

FIG. 4 shows a time course study of fed-batch fermentation of Bacillus subtilis bFGF according to one embodiment of the present invention. Culture samples were obtained at different time points before and after IPTG induction. FIG. 4A: Western blot analysis results of bFGF present in cell lysate (CL) samples, where 5 µl of cell lysate was loaded per lane. FIG. 4B: cell viability and quantification of bFGF. (--•--) indicates detectable bFGF levels; CFU refers to colony forming units. The viable cell counts were determined on common agar plates and plates with kanamycin, and are represented by (-•-) and (-■-), respectively. Transformant growth experiments were repeated in triplicate and standard error bars are shown.

FIG. 5 shows mass spectrometry results (molecular size) of purified bFGF samples derived from the pECBS1-H6-DnaE-bFGF construct according to one embodiment of the present invention.

FIG. 6 shows the experimental results of mitogenic activity of bFGF protein according to one embodiment of the present invention. The effect of different concentrations of purified bFGF protein samples derived from the pECBS1-H6-DnaE-bFGF construct on fibroblast proliferation is shown.

FIG. 7 shows the results of restriction digestion identification of the pECBS1-H6-DnaE-bFGF construct.

FIG. 8 shows WB results of bFGF protein expression using H6 and CBD affinity tags, respectively. Lanes 0 h, 4 h, 8 h: represent samples collected from the cultures at 0 h, 4 h, and 8 h after induction, respectively; lane+ve and lane-ve represent positive and negative controls, respectively.

FIG. 9 shows WB results of bFGF protein expression using H6 and GST affinity tags, respectively. Lanes 0 h, 4 h, 8 h: represent samples collected from the cultures at 0 h, 4 h, and 8 h after induction, respectively; lane+ve and lane-ve represent positive and negative controls, respectively.

FIG. 10 shows the results of purification only by use heparin-agarose chromatography.

### EXAMPLES

The following provides a description of expression systems and methods that can be used to express a variety of exogenous polypeptides, particularly native exogenous polypeptides. These systems and methods satisfy at least one need existing in the art.

The section headings used herein are for organizational purposes only and should not be construed as limiting the subject matter in any way.

Unless explicitly defined otherwise, the terms used herein are to be understood according to their ordinary meanings in the art. Unless otherwise specified or indicated by the context, nouns without quantifier modifiers mean one or more.

Standard techniques and procedures are generally performed according to conventional methods in the art and various general references (see generally, Sambrook et al. Molecular Cloning: A Laboratory Manual, 2nd ed. (1989) Cold Spring Harbor Laboratory Press, Cold Spring Harbor, NY).

In one embodiment, the present invention provides a nucleic acid construct, which comprises an insert, and the insert comprises, from the 5′ end to the 3′ end, a polynucleotide sequence encoding a short peptide affinity tag, a trans-splicing intein derived from Anabaena sp., and an exogenous polypeptide, and wherein the short peptide affinity tag serves as an N-terminal extein of the trans-splicing intein, and the exogenous polypeptide serves as a C-terminal extein of the trans-splicing intein.

It should be noted that the embodiments in the present application and the features of the embodiments can be combined with each other under the condition of no conflict. Intein

Intein is a protein element capable of self-cleavage from a host protein, and its catalytic flanking sequences are linked by peptide bonds.

Intein useful in the present invention may be trans-splicing intein derived from Anabaena sp. In one embodiment, the intein may be an intein (Asp DnaE) derived from an Anabaena DNA polymerase III unit.

As used herein, the term “trans-splicing intein” refers to an intein having trans-splicing activity. According to the form in which they exist, intein can be divided into whole intein and split intein. The two former splicing regions coexist on the same polypeptide fragment, while the two latter splicing regions exist on different polypeptide fragments, which is called split intein. Whole intein undergo cis-splicing, while split intein undergo trans-splicing. A split intein may also be referred to as a trans-splicing intein.

As used herein, the term “intein of Anabaena DNA polymerase III unit” refers to intein derived from an Anabaena DNA polymerase III unit. In one aspect, the nucleotides encoding the inteins of the invention may have or comprise the sequence of SEQ ID NO: 1 or a complementary sequence thereof or may have or comprise a sequence of SEQ ID NO: 1 with at least 75%, 80%, 85%, 90%, 95%, 96%, 97%, 98%, 99% or more sequence identity or its complementary sequence, or may be composed of the above nucleotides sequences. In one aspect, the intein may have or comprise the amino acid sequence of SEQ ID NO: 2 or may have or comprise a sequence of SEQ ID NO: 2 with at least 75%, 80%, 85%, 90%, 95%, 96%, 97%, 98%, 99% or more sequence identity, or may be composed of the above amino acid sequences.

### Exogenous Polypeptide

As used herein, the terms “foreign polypeptide”, “exogenous protein”, “heterologous polypeptide” and “heterologous protein” are used interchangeably and refer to a polypeptide or protein that is not naturally expressed by a host cell, but is artificially added or expressed by a host cell by techniques such as gene transfection.

In some embodiments, the heterologous polypeptide can be, for example, an enzyme, a cytokine (e.g., fibroblast growth factor), a hormone (e.g., calcitonin, erythropoietin, thrombopoietin, human growth hormone, epidermal growth factor) etc.), interferons, or other proteins with therapeutic, nutraceutical, agricultural or industrial uses. Additional heterologous polypeptides can be antibodies, antibody fragments, and drug proteins. A heterologous polypeptide can also be a polypeptide fragment.

In one embodiment, the heterologous polypeptide useful in the present invention may be a fibroblast growth factor (FGF). Fibroblast growth factors are a class of polypeptides consisting of about 150-200 amino acids that exist in two closely related forms, basic fibroblast growth factor (bFGF) and acidic fibroblast growth factor (aFGF).

In one embodiment, the heterologous polypeptide useful in the present invention may be a basic fibroblast growth factor, especially human bFGF, more particularly native human bFGF. In one aspect, the bFGF of the invention may have or comprise the nucleotide sequence of SEQ ID NO: 3 or may have or comprise a sequence of SEQ ID NO: 3 with at least 75%, 80%, 85%, 90%, 95%, 96%, 97%, 98%, 99% or more sequence identity, or may be composed of the above amino acid sequences.

In one aspect, the bFGF of the present invention may have or comprise the amino acid sequence of SEQ ID NO: 4 or may have or comprise a sequence of SEQ ID NO: 4 with at least 75%, 80%, 85%, 90%, 95%, 96%, 97%, 98%, 99% or more sequence identity, or may be composed of the above amino acid sequences.

### Affinity Tag

As used herein, the terms “affinity tag”, “purification tag” and “protein tag” are used interchangeably and refer to a protein or polypeptide expressed in fusion with a target protein during recombinant protein preparation. An affinity tag can be used to promote the solubility and stability of the target protein and facilitate the detection and purification of the target protein.

Without intending to be limited by theory, the present inventors have unexpectedly discovered that short peptide affinity tags of relatively small molecular weight, lower than obtaining mature and biologically identical (native) foreign proteins or polypeptides, are beneficial.

In some embodiments, the affinity tag useful in the present invention may be a short peptide affinity tag, which may have about 4-15 amino acids in length, e.g., 5, 6, 7, 8, 9, 10, 11, 12, 13, 14 or 15 amino acids. In some embodiments, the short peptide affinity tags include, but are not limited to: HIS tags, HA tags (e.g., YPYDVP), FLAG tags (e.g. DYKDDDDK), HSV tags (e.g. QPELAPEDPED), MYC tags (e.g. ILKKATAYIL or EQKLISEEDL), V5 tags (e.g. GKPIPNPLLGLDST), Xpress tags (e.g. DLDDDDK or DLYDDDDK), Thrombin tags (e.g. LVPRGS), BAD (biotin receptor domain) (e.g. GLNDIFEAQKIEWHE), Factor Xa tags (e.g. IEGR or IDGR), VSVG tags (e.g. YTDIEMNRLGK)), SV40 NLS tags (such as PKKKRKV or PKKKRKVG), protein C tags (such as EDQVDPRLIDGK), S tags (such as KETAAAKFERQHMDS), SB 1 tags (such as PRPSNKRLQQ), etc. In one aspect, the affinity tag may be a 5-15 x His tag, more specifically a 6 x His tag (H6).

In some embodiments, the short peptide affinity tag of the invention serves as the N-terminal extein of a trans-splicing intein, and the exogenous polypeptide serves as the C-terminal extein of the trans-splicing intein. In one example, the H6 tag is fused to the N-terminus of the Asp DnaE intein, and bFGF is fused to the C-terminus of the Asp DnaE intein.

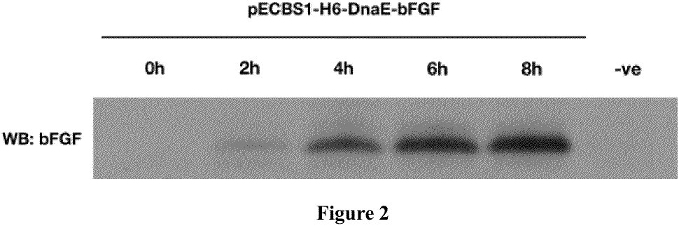

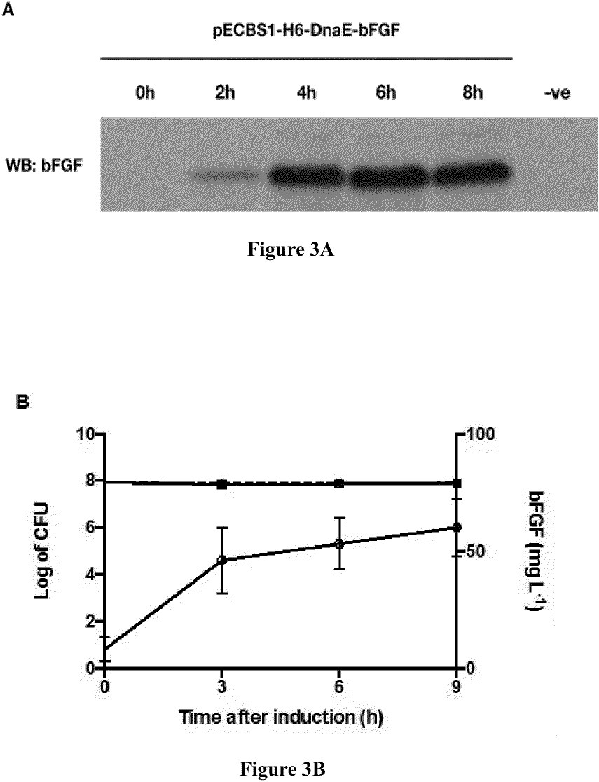

The coding sequence of bFGF was first designed to be fused to the C-terminus of the intein, and the short peptide affinity tag served as an anchor for protein purification after expression. From the experimental results, fusing a GST or CBD tag with a larger size to the N-terminus of the DnaE intein only results in a precursor aggregated form, while replacing the N-segment extein with a short peptide affinity tag with a smaller size such as H6 tag gets surprising results. The expressed bFGF was not only in the solubilized form but also in the mature state and had high yields (see FIGS. 2, 3A and 3B). Without intending to be limited by theory, the replacement of the N-terminal extein with a relatively short peptide affinity tag may have altered the overall conformation of the entire fusion protein, thereby facilitating isolation of the C-terminal extein and avoiding inclusion bodies Formation.

### Expression Vector

In one embodiment, the present invention provides an expression vector comprising the nucleic acid construct of the present invention.

As used herein, the terms “vector”, “expression vector”, “recombinant vector” and “recombinant system” are used interchangeably and refer to a vehicle, through which a polynucleotide or DNA molecule can be manipulated or introduced into host cell. The vector may be a linear or circular polynucleotide or may be a large size polynucleotide or any other type of construct, such as DNA or RNA from a viral genome, virion, or any other biological construct, it allows the manipulation of DNA or its introduction into cells.

Those skilled in the art will appreciate that there is no limit to the type of vector that can be used, so long as the vector can be a cloning vector suitable for propagation, capable of obtaining sufficient polynucleotides or genetic constructs, or an expression vector suitable for purification of fusion proteins in different heterologous organisms. In one embodiment, suitable vectors according to the invention include expression vectors in prokaryotes, such as prokaryotic expression vectors, including but not limited to: pET14, pET21, pET22, pET28, pET42, pMAL-2c, pTYB2, pGEX-4T-2. pGEX-6T-1, pQE-9, pBAD-his, pBAD-Myc, pECB series vectors, pRB series vectors, etc., such as pUC18, pUC19, Bluescript and its derivatives, mp18, mp19, pBR322, pBR374, pMB9, CoIE1, pCR1, RP4, phage and “shuttle” vectors (e.g., pSA3 and pAT28).

In one embodiment, the present invention also contemplates shuttle vectors. As used herein, the term “shuttle vector” is a class of vectors that can replicate and amplify in two different host cells (e.g., E. coli and B. subtilis), thereby enabling transformation of the same expression vector into different host cells. The shuttle vectors involved in the present invention may include but are not limited to pECBS1.

Vector components may generally include, but are not limited to, one or more of the following expression control elements: promoters, enhancers, operators, ribosome binding sites, transcription termination sequences, and the like.

Exemplary promoters useful in the present invention may include promoters active in prokaryotes, such as T7 promoter, phoA promoter, β-lactamase and lactose promoter system, alkaline phosphatase, tryptophan (trp) promoter system, and hybrid promoters such as the tac promoter.

Exemplary operons useful in the present invention include, but are not limited to, lactose operon, arabinose operon, tryptophan operon, and the like. The lactose operon is a group of genes involved in the breakdown of lactose, consisting of repressor and operator sequences of the lactose system, so that a group of genes related to lactose metabolism are synchronously regulated.

As used herein, the term “ribosome binding site” (RBS) refers to a sequence located upstream of the start codon of mRNA that is available for binding to a ribosome at the initiation of translation.

The expression vector according to the present invention may further comprise a polynucleotide encoding a marker protein. Marker proteins suitable for the present invention include those proteins with antibiotic resistance or resistance to other toxic compounds. Examples of marker proteins with antibiotic resistance include neomycin phosphotransferase to phosphorylate neomycin and kanamycin, or hpt to phosphorylate hygromycin, or to confer resistance to, for example, bleomycin, streptomycin, tetracycline, chloramphenicol, ampicillin, gentamicin, geneticin (G418), spectinomycin or blasticidin-resistant proteins. In one example, the protein confers resistance to chloramphenicol. For example, the protein is a gene from E. coli designated CmR as described in Nilsen et al, J. Bacteriol, 178:3188-3193, 1996.

A polynucleotide encoding target polypeptide is cloned into a vector of the invention using standard techniques well known to those skilled in the art. For example, the polymerase chain reaction (PCR) is used to generate polynucleotides encoding the target polypeptides. PCR procedures are known in the art.

In some embodiments, the nucleic acid constructs of the present invention may further comprise a first cloning site upstream of the insert and a second cloning site downstream of the insert, wherein the first cloning site and the second cloning site allow insertion of the nucleic acid construct into the expression vector.

Cloning sites allow cloning of polynucleotides encoding heterologous polypeptides. Preferably, the cloning sites are grouped together to form multiple cloning sites. As used herein, the term “multiple cloning site” refers to a nucleic acid sequence comprising a series of two or more restriction endonuclease target sequences located adjacent to each other. Multiple cloning sites contain restriction endonuclease targets that allow insertion of fragments with blunt ends, sticky 5′ ends or sticky 3′ ends. Insertion of a polynucleotide of interest is performed using standard molecular biology methods, e.g., as described by Sambrook et al. (Sambrook et al. Molecular Cloning: A Laboratory Manual, Cold Spring Harbour Laboratory Press, 1989) and/or Ausubel et al. (Current Protocols in Molecular Biology, Greene Pub. Associates and Wiley-Interscience, 1988).

As used herein, the term “restriction endonuclease” or “restriction endonuclease” refers to a class of enzymes that can recognize and attach specific deoxynucleotide sequences and cleave the phosphodiester bond between two deoxyribonucleotides at specific sites in each chain. The cleavage method is to cut off the bond between the sugar molecule and the phosphate, and then create a nick on each of the two DNA strands without destroying the nucleotides and bases. There are two forms of cleavage, sticky ends that may produce protruding single-stranded DNA, and blunt ends that have flat ends without bulges. The broken DNA fragments can be joined by DNA ligase, therefore different restriction fragments in chromosomes or DNA can be joined together by splicing. Restriction enzymes that can be used in the present invention may include, but are not limited to, EcoRI, PstI, XbaI, BamHI, HindIII, TaqI, NotI, HinjI, Sau3A, PovII, SmaI, HaeIII, AluI, SalI, Dra and the like.

Methods of ligating nucleic acids are apparent to those skilled in the art, and are described, for example, in Sambrook et al. Molecular Cloning: A Laboratory Manual, Cold Spring Harbour Laboratory Press, 1989 and/or Ausubel et al. (eds.), Current Protocols in Molecular Biology, Greene Pub. Associates and Wiley-Interscience (1988). In one example, nucleic acids are ligated using a ligase (e.g., T4 DNA ligase).

### Host Cell

In some embodiments, the present invention provides a transformed host cell comprising the expression vector of the present invention. Bacillus subtilis is considered “generally recognized as safe” because it does not contain endotoxins, in one embodiment of the present invention, Bacillus subtilis is used as a host cell. The inventors of the present invention have surprisingly found that Bacillus subtilis using the nucleic acid construct structure of the present invention can promote the automatic cleavage of intein and extein, and it can obtain satisfactory expression levels of native exogenous polypeptides/proteins and the problem of low expression level of exogenous polypeptide/protein expressed by Bacillus subtilis is solved.

In some respects, the invention provides a method of obtaining a transformed Bacillus subtilis, comprising contacting the Bacillus subtilis with an expression vector of the invention under conditions that allow transformation of the expression vector into Bacillus subtilis. Those skilled in the art know and can adjust suitable conditions depending on the type of expression vector and host cell.

As used herein, the term “transformation” means the introduction of DNA into a prokaryotic host as an extrachromosomal element or by chromosomal integration, such that the DNA can be replicated. Depending on the host cell used, transformation is performed using standard techniques appropriate for such cells. Calcium treatment with calcium chloride is typically used for bacterial cells containing a strong cell wall barrier. Another method for transformation uses polyethylene glycol/DMSO. Another technique also used is electroporation.

The prokaryotic host cells used to produce the exogenous polypeptides of the invention are cultured in media known in the art and suitable for use in the culture of the host cells. An example of a suitable medium may include Luria-Bertani (LB) medium with essential nutrient supplements. In some embodiments, the culture medium further contains a selection agent, based on the constructed expression vector to select, selectively allowing the growth of prokaryotic cells containing the expression vector. For example, ampicillin and/or kanamycin are added to the medium for cell growth, the cells express ampicillin and/or kanamycin resistance genes. Any necessary supplements other than carbon, nitrogen and inorganic phosphorus sources may also be included at suitable concentrations, either alone or in admixture with another supplement or medium such as a complex nitrogen source.

### Method for Producing Exogenous Polypeptide

In some embodiments, the present invention provides a method of producing an exogenous polypeptide comprising culturing a transformed Bacillus subtilis of the present invention under conditions that permit expression of the exogenous polypeptide.

For accumulation of the expressed gene product, the host cell is cultured under conditions sufficient to accumulate the gene product. Such conditions can include, for example, temperature, nutritional and cell density conditions that allow cells to express and accumulate proteins. Furthermore, as known to those skilled in the art, such conditions are those under which the cell can perform essential cellular functions such as transcription, translation, and intracellular expression.

Prokaryotic host cells were cultured at appropriate temperature. For Bacillus subtilis culture, for example, the general temperature is about 20° C. to about 39° C. In one embodiment, the temperature is about 25° C. to about 37° C., such as 37° C.

For induction, cells are typically cultured until a defined optical density is reached, e.g., about 80-100 A55tl, at which point induction begins (e.g., by adding inducers, by depleting repressors, inhibitors, or media components, etc.), to induce expression of a gene encoding a heterologous polypeptide.

After the product has accumulated, the cells present in the culture can be mechanically lysed using any mechanical method known in the art to release the protein from the host cell. Optionally, other cleavage methods can also be used, including but not limited to alkaline cleavage methods, SDS cleavage methods, and the like. Cell lysates used to lyse cells may include, but are not limited to, Tris-HCl, EDTA, NaCl, glucose, lysozyme, and the like. The lysate is incubated for a sufficient time to release the heterologous polypeptide contained in the cells, optionally prior to product recovery.

The lysate can be subjected to further processing such as dilution with water, addition of buffers or flocculants, pH adjustment, or changing or maintaining the temperature of the lysate/homogenate in the preparation for subsequent recovery steps.

In the subsequent steps, the heterologous polypeptide is recovered from the lysate in a manner that minimizes co-recovered cellular debris and products. Recycling can be done by any method. In one embodiment, settling the heterologous polypeptide-containing refract able particles or collecting the soluble product-containing supernatant may be included. An example of sedimentation can be centrifugation. Recovery can be performed in the presence of an agent that disrupts the outer cell wall to increase permeability and allow recovery of more solids prior to adsorption or sedimentation. Examples of such agents include chelating agents such as ethylenediaminetetraacetic acid (EDTA) or zwitterionic such as dipolar ionic detergents such as ZWITTERGENT 316™ detergent. In one embodiment, recovery is performed in the presence of EDTA.

In one embodiment, if desired, it may further comprise isolating the aggregated heterologous polypeptide, followed by simultaneous solubilization and refolding of the polypeptide. Alternatively, the soluble product can be recovered by standard techniques as described below: fractionation on an immunoaffinity or ion exchange column; ethanol precipitation; reverse phase HPLC; chromatography on silica or on a cation exchange resin such as DEAE; chromatographic focusing; SDS-PAGE; ammonium sulfate precipitation; and gel filtration using e.g., SEPHADEX™ G-75; heparin-agarose (HA) chromatography, etc.

In one embodiment, the method of producing an exogenous polypeptide of the present invention further comprises purifying the exogenous polypeptide from the cell lysate by sequentially employing cation exchange chromatography and heparin-agarose (HA) chromatography. The inventors have surprisingly found that before using heparin agarose chromatography, cation exchange chromatography was used to remove most housekeeping proteins, and then high concentration of salt was removed by dialysis to obtain unexpected high purity bFGF protein.

Although B. subtilis is an attractive host system for protein production due to its absence of endotoxin, current studies have demonstrated difficulty in obtaining high levels of intracellular expression of soluble heterologous proteins. Many researchers have developed intein and its application in protein expression, however the mechanism of action of intein in different host systems is still not fully understood. The inventors attempted to express endotoxin-free recombinant proteins in bacterial host systems for research and commercial purposes. Compared to other methods, expressing protein using intein is the easiest and most economical method to produce recombinant proteins with biologically identical structures, ensuring high biological activity and preventing adverse immune responses in animal subjects.

To overcome the defects of the prior art and improve the predictability and effectiveness of the intein-mediated protein purification system, the inventors constructed a brand-new protein expression and purification system. This system utilizes an intein (Asp DnaE) derived from Anabaena species, especially the DNA polymerase III unit, to facilitate the intracellular expression of foreign proteins such as human bFGF protein in Bacillus subtilis, and by adding a short peptide affinity tag (such as 6 x His tag) to the N-terminal of the fusion protein, the efficiency of protein purification can be improved, and the active natural foreign protein (such as human bFGF protein) can be obtained. Using the construct provided by the present invention to express exogenous protein such as human bFGF can significantly improve the expression efficiency of biologically active exogenous protein such as human bFGF protein, reduce the generation of inclusion bodies, simplify the purification steps, and greatly reduce the purification cost. The inventors also experimentally confirmed that the construct and protein purification system of the present invention significantly increased the total production of exogenous proteins such as human bFGF protein in a 4 L scale fermentation experiment compared with shake flask culture, while cell viability was maintained throughout the induction period. It is also stable, especially suitable for large-scale cultivation, and has achieved unexpected technical effects.

Exemplary sequences in the present invention are shown in the table below.

| | |
|---|---|
| Sequence number | Nucleotide/amino acid sequence |
| SEQ ID NO: 1 | ATGATTAAAATTGCGAGCCGCAAATTTCTGGGCGTGGAAAACGTGTATGATATTGGCGTGCGCCGCGATCATAACTTTTTTATTAAAAACGGCCTGATTGCGAGCAAC |
| SEQ ID NO: 2 | MIKIASRKFLGVENVYDIGVRRDHNFFIKNGLIASN |
| SEQ ID NO: 3 | CCAGCCTTGCCAGAGGATGGCGGCAGCGGCGCCTTCCCGCCAGGCCACTTCAAGGACCCAAAGCGCCTGTACTGCAAAAACGGGGGCTTCTTCCTGCGCATCCACCCAGACGGCCGCGTTGACGGGGTCCGCGAGAAGAGCGACCCTCACATCAAGCTACAACTTCAAGCAGAAGAGCGCGGAGTTGTGTCTATCAAAGGAGTGTGTGCTAACCGTTACCTGGCTATGAAGGAAGATGGACGCTTACTGGCTTCTAAATGTGTTACGGATGAGTGTTTCTTTTTTGAACGCTTGGAATCTAATAACTACAATACTTACCGCTCACGCAAATACACCAGTTGGTATGTGGCACTGAAACGCACTGGGCAGTATAAACTTGGATCCAAAACAGGACCTGGGCAGAAAGCTATCCTTTTTCTTCCAATGTCTGCTAAGAGC |
| SEQ ID NO: 4 | PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS |
| SEQ ID NO: 5 | GAATTCTAATACGACTCACTATAGGGAGATTGTGAGCGGATAACAATTTGTTTAACTTTAAGAAGGAGAATGCATCATCACCATCACCACATGATTAAAATTGCGAGCCGCAAATTTCTGGGCGTGGAAAACGTGTATGATATTGGCGTGCGCCGCGATCATAACTTTTTTATTAAAAACGGCCTGATTGC GAGCAACCCAGCCTTGCCAGAGGATGGCGGCAGCGGCGCCTTCCCGCCAGGCCACTTCAAGGACCCAAAGCGCCT GTACTGCAAAAACGGGGGCTTCTTCCTGCGCATCCACCCAGACGGCCGCGTTGACGGGGTCCGCGAGAAGAGCGACCCTCACATCAAGCTACAACTTCAAGCAGAAGAGCGCGGAGTTGTGTCTATCAAAGGAGTGTGTGCTAACCGTTACCTGGCTATGAAGGAAGATGGACGCTTACTGGCTTCTAA ATGTGTTACGGATGAGTGTTTCTTTTTTGAACGCTTGGAATCTAATAACTACAATACTTACCGCTCACGCAAATACACCAGTTGGTATGTGGCACTGAAACGCACTGGGCAGTATAAACTTGGATCCAAAACAGGACCTGGGCAGAAAGCTATCCTTTTTCTTCCAATGTCTGCTAAGAGCTAAAGACCCG GGGCTTAATTAATTAAGCTAGCATAACCCCTTGGGGCCTCTAAACGGGTCTTGAGGGGTTTTTTGTCTAGA |

### EXAMPLE

The present invention is described herein by the following examples, which are intended to be illustrative only and not intended to limit the scope of the present invention.

E. coli strain DH5α was purchased from New England Biolabs (Ipswich, MA). Bacillus subtilis strain WB800 was obtained as described in a previous report [13]. Synthetic DNA fragments, restriction enzymes and antibodies against bFGF were purchased from Thermo Fisher Scientific (Ipswich, MA). Unless otherwise stated, all other chemicals were purchased from Sigma-Aldrich (St. Louis, MO).

### Example 1: Expression Vector Construction and Host Cell Transformation

### Construction and Design of Escherichia Coli/Bacillus Subtilis Expression Shuttle Vector

pRB374 and pBR322 were used as starting vectors for the E. coli/ B. subtilis expression shuttle vectors, respectively [14]. Specifically, pECBS1 was constructed by the following modification steps: first, pRB374 (5.9 kb) was digested with SalI and BglII; after both sites were digested with the same SalI and BglII, the T7 ribonucleic acid polymerase-Lac promoter-LacI gene-LacI^{q} promoter-bleomycin resistance gene-part of the neomycin resistance gene fragment (5.3 kb) was replaced to form pECBSi vector. Then, the resulting pECBSi vector and pBR322 vector were digested with EcoRI and BglI, respectively, and the pECBSi digested fragment was replaced with a fragment obtained by digesting pBR322 (4.3 kb), thereby forming the pECBS1 shuttle vector.

### Construction of bFGF Expression Vector

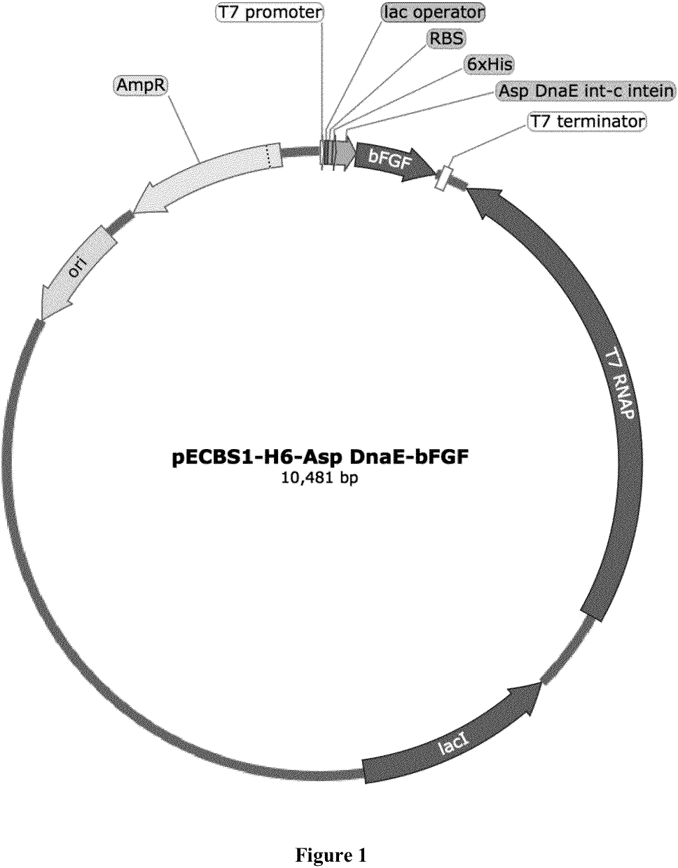

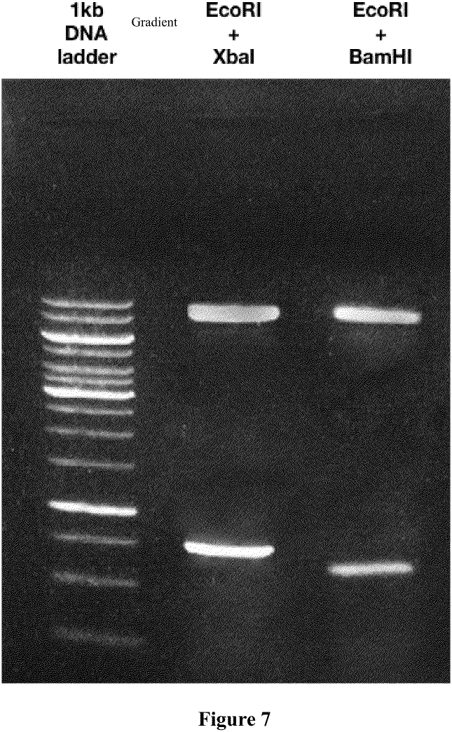

The construction method of E. coli/Bacillus subtilis expression shuttle vector (pECBS1-H6-DnaE-bFGF) is as follows: a DNA fragment coding EcoRI-T7 promoter (T7)-lactose operon (LacO)-ribosome binding site (RBS)-6x-His-tag (H6)-Asp-DnaE int-c (DnaE)-bFGF-T7 transcription terminator-Xbal sequence was synthesized by Thermo Fisher Scientific, shown in SEQ ID NO: 5. The previously synthesized DNA fragment was digested with EcoRI and XbaI, and then the Bacillus subtilis/E. coli shuttle vector pECBS1 was ligated with the same two restriction enzymes. The pECBSl-H6-DnaE-bFGF construct was finally obtained (see FIG. 1). The results of restriction enzyme cleavage identification of the obtained constructs are shown in FIG. 7.

### Transformation of Bacillus Subtilis

A single colony of WB800 was inoculated into 5 ml of Medium A (containing 1x Spizizen’s salt solution, 0.5% glucose, 0.005% tryptophan, 0.02% casamino acids, 0.5% yeast extract, 0.8% arginine, 0.4% group acid) and incubated at 37° C., 200 rpm overnight. 0.5 ml of the overnight culture was then subcultured into 50 ml of medium A and incubated at 37° C., 200 rpm until A600 = 1.7. 1 ml of 87% glycerol was added to 10 ml of the culture and placed on ice for 15 minutes. 1 ml of the culture was then further subcultured to 20 ml medium B (containing 1x Spizizen salts, 0.5% glucose, 0.0005% tryptophan, 0.01% casamino acids, 0.1% yeast extract, 2.5 mM MgCl₂, 0.5 mM CaCl₂) and incubated at 30° C., 150 rpm for 2 hours. 1 ml of the culture was transferred to a microcentrifuge tube and EGTA was added at a final concentration of 1 mM and incubated for 5 min at room temperature. 2 ug of plasmid DNA was then added to 1 ml of competent WB800 and allowed to continue to grow for 2 hours at 37° C., 200 rpm. Transformed WB800 was then collected by centrifugation at 5000 rpm at room temperature and resuspended in 100 ul of culture supernatant. Transformed WB800 was plated on kanamycin resistant plates and incubated overnight at 37° C.

### Example 2: Expression of bFGF

### Shake Flask Culture

B. subtilis transformants were grown in 200 ml 2x LB medium with 25 µg/ml kanamycin at 37° C. (250 rpm) [15]. When the A600 value reached 1.0, IPTG was added to a final concentration of 0.2 mM, followed by 1 ml culture samples collected every 3 h intervals for bFGF expression analysis. The cell pellet was resuspended in 200 µl of resuspension buffer (50 mM Tris-Cl, 200 mM EDTA, pH 8.0), then incubated on ice for 5 min. The mixture was then treated with 120 µl of lysozyme solution (10 mg/mL) for 20 min at 37° C. Then 80 µl of lysis buffer (10 mM EDTA, 10% Triton X-100 and 50 mM Tris-Cl, pH 8.0) was added. The tube containing the solution was gently inverted and centrifuged at 14,800 rpm for 5 minutes. Cell lysate samples were analyzed for bFGF protein expression by Western blotting.

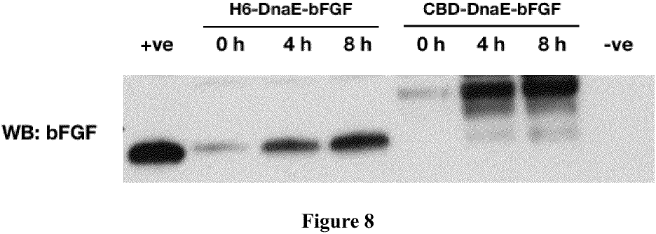

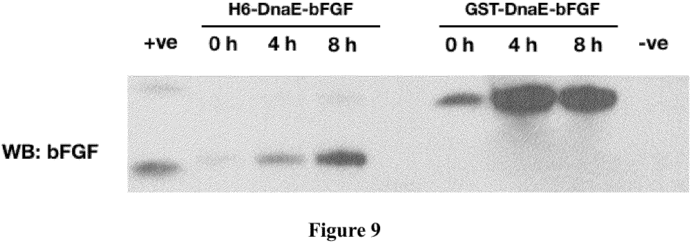

To successfully express the soluble bFGF protein, the inventors also tested the combination of different inteins and exogenous polypeptides through experiments, and finally found that the Asp DnaE intein is beneficial. bFGF was chosen to be fused to the Asp DnaE intein C-terminus, because in vitro cleavage at the C-terminus of DnaE can be controlled by pH changes or reducing agent treatment. Furthermore, the inventors tried several different expression tags, including GST, chitin binding domain (CBD) and H6 affinity tag. With the first two expression tags, the construct yielded only the insoluble form of the precursor (see FIGS. 8 and 9), while with the relatively small size H6 tag, the experimental results gave positive results for mature and biologically identical bFGF expression results (see Table 1 for details). The results of the shake flask culture experiments showed (see FIG. 2) that the final product bFGF expressed by the construct pECBS1-H6-DnaE-bFGF upon induction was at satisfactory levels, and no precursor form was detected from the Western blot (see FIG. 8 and FIG. 9).

TABLE 1

Analysis of purified bFGF by liquid chromatography-mass spectrometry

| Construct | Peptide^{a} | Theoretical mass-to-charge ratio of peptides | Experimental mass-to-charge ratio of peptides | ion score |
|---|---|---|---|---|
| pECBS1-H6-Asp DnaE-bFGF | PALPEDGGSGAFPPGHFKD | 1779 | 1781.5 | 46 |
| | RLYCKNGGFFLRI | 1374 | 1373.5 | 25 |
| | RIHPDGRVDGVRE | 1220 | 1220.2 | 23 |
| | KLQLQAEERG | 985 | 985.8 | 59 |
| | RGVVSIKGVCANRY | 1259 | 1258.2 | 34 |
| | RYLAMKEDGRL | 1098 | 1098.3 | 44 |
| | RLLASKCVTDECFFFERL | 2021 | 2020.6 | 36 |
| | RLESNNYNTYRS | 1273 | 1272.2 | 63 |
| | RKYTSWYVALKR | 1257 | 1257.3 | 57 |
| | KAILFLPMSAKS- | 1105 | 1106 | 52 |

a. after partial trypsinization of purified bFGF, N-terminal and C-terminal sequences were identified by Mascot search engine

### Fed-Batch Fermentation

B. subtilis transformants were grown in 200 ml 2x LB medium with 25 µg/ml kanamycin at 37° C. (250 rpm) until A600 = 1.0. 50 ml of the culture was then transferred to a 2 L flask (containing 450 ml of 2x LB medium with 25 µg/ml kanamycin), and the culture was continued at 37° C. (rotation 250 rpm) until the A600 value achieving 1.0. The entire culture was inoculated into a 5 L fermenter containing 3.5 L of 2x LB medium with 25 µg/ml kanamycin, and the pH value of the culture was maintained at 7.0 by adding 1 M NaOH. The pO₂ value (partial pressure of oxygen) of the culture was set at 1.5 vvm. In addition, when the pH value started to rise, a 50% glucose feed solution was added to maintain the pH of the culture at 7.0. When A600 = 8, IPTG was added at a final concentration of 0.2 mM for induction culture. pH adjustment was maintained with 1 M H₂SO₄. Culture samples were collected at 2 hours intervals for analysis of bFGF expression.

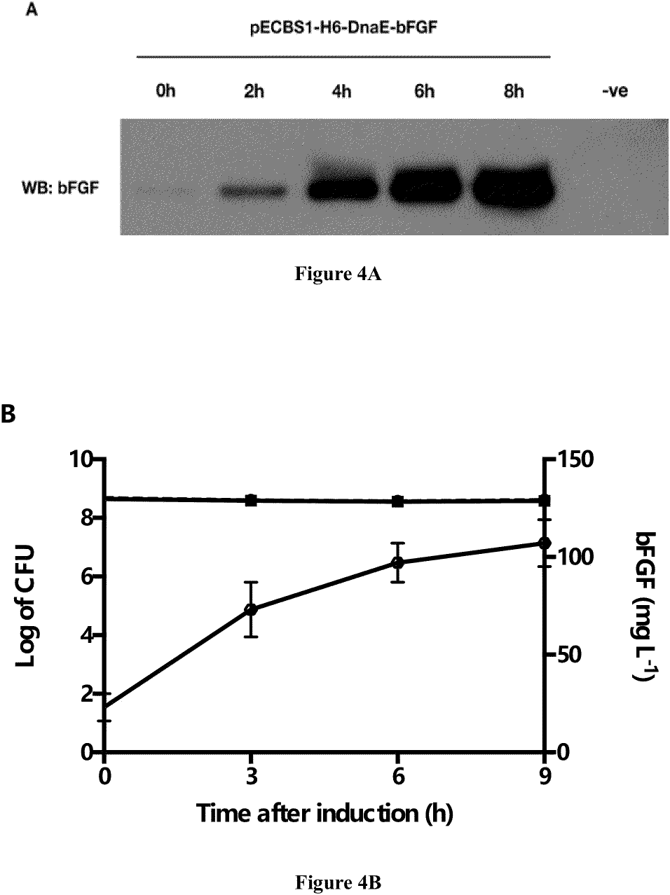

The results showed that the bFGF protein expression and cell mass of Bacillus subtilis were significantly increased. Specifically, the total bFGF protein production and the final colony-forming units (CFU) of the expression constructs increased 2-fold (from 64 mg/L to 113 mg/L) and 6-fold respectively from shake flask culture (FIG. 3) to large-scale culture (FIG. 4). Thus, the construct obtained by the present invention achieves unexpected technical effects in both shake flask culture and large-scale fermentation culture.

### Example 3: Purification and Structural Determination of bFGF Protein

Cation exchange chromatography and heparin-agarose chromatography were used to purify bFGF. First, the protein concentration of the eluted fractions was measured using a Nanodrop Microvolume spectrophotometer. In addition, the eluted fractions with significant readings (approximately 1 mg/ml) were combined and dialyzed against 0.1x PB. Afterwards, purified bFGF bands were obtained by electrophoresis on a 10% SDS-PAGE gel stained with Coomassie brilliant blue R-250. Bands containing bFGF protein from the SDS-PAGE gel were recovered for subsequent analysis by LC-MS.

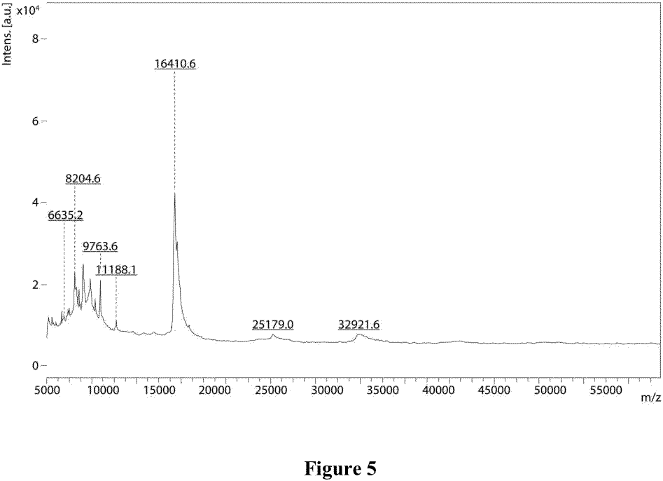

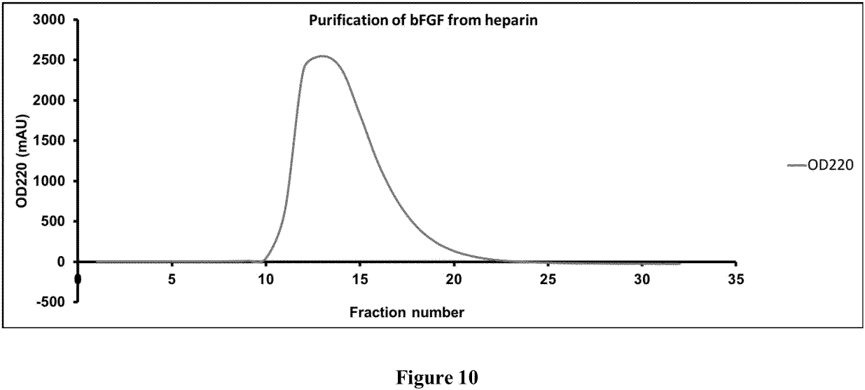

Western blot analysis showed that the soluble bFGF protein extracted from the lysate had the same molecular weight as the bFGF protein purchased from Thermofisher Scientific (FIG. 4). The purified bFGF protein samples were subjected to N-terminal and C-terminal protein sequencing and MALDI-TOF quality determination by LC-MS. The results showed that the final bFGF protein product obtained from the expression of the H6-DnaE-bFGF construct had a biological structure of 146 amino acids (Table 1) and a size of 16.4 kda (FIG. 5), which was consistent with the native human bFGF protein. Compared with the purification by only heparin-agarose chromatography (FIG. 10), cation-exchange chromatography and heparin-agarose chromatography were adopted in the present invention to obtain high-purity bFGF protein.

### Example 4: Detection of Biological Activity of bFGF Protein

The effect of purified bFGF protein on the proliferation of NIH/3T3 fibroblasts was detected using MTT assay (also known as MTT colorimetry). The specific steps are as follows: NIH/3T3 cells (the density is 2 × 10⁴ cells) were inoculated in a 96 well plate, starved for 24 hours in DMEM medium with 1% fetal bovine serum at 37° C., 5% CO₂, and then treated with bFGF at different concentrations for 3 days. 3-(4,5-Dimethylthiazol-2-yl)-2,5-diphenyltetrazolium bromide (MTT) at a final concentration of 0.5 mg/ml was added to each well of the plate and incubated at 37° C. and 5% CO₂ for 4 hours. Then all the solution in the wells were removed and 150 µL DMSO was added to dissolves the purple crystals. The plate was shaken continuously in the dark for 10 minutes, and the absorbance was read with an enzyme labeling instrument at 570 nm.

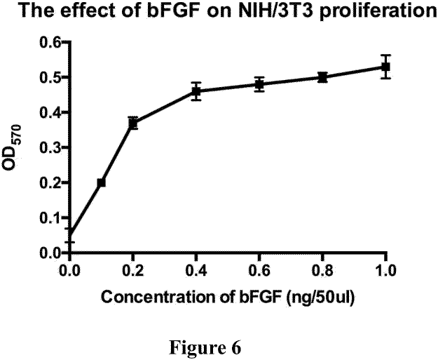

The results showed that the purified bFGF protein expressed in Bacillus subtilis was able to induce NIH/3T3 cells proliferation (FIG. 6) and human mesenchymal stem cells (data not shown). Thus, the purified bFGF protein obtained by the present invention has biological activity (mitogenic activity).

The above results show that the purified bFGF protein of the present invention has the same primary sequence of 146 amino acids as the wild protein, it is a mature soluble protein form and has high biological activity in inducing the proliferation of NIH/3T3 cells. In addition, the inventors also tried different scales of fermentation culture, and unexpected technical effects were obtained in terms of bFGF protein expression and cell mass.

Those skilled in the art will further realize that the present invention may be embodied in other specific forms without departing from its spirit or central characteristics. Since the foregoing description of this invention discloses only exemplary embodiments thereof, it is to be understood that other variations are within the scope of this invention. Therefore, the invention is not limited to the specific embodiments described in detail herein. Rather, reference should be made to the appended claims to indicate the scope and content of the invention.

### REFERENCE

[1] Levenstein, M. E., Ludwig, T. E., Xu, R. H., Llanas, R. A., Van Den Heuvel -Kramer, K., Manning, D., & Thomson, J. A. (2006). Basic fibroblast growth factor support of human embryonic stem cell self - renewal. Stem cells, 24(3), 568-574.
[2] Xu, C., Rosler, E., Jiang, J., Lebkowski, J. S., Gold, J. D., O’Sullivan, C., ... & Carpenter, M. K. (2005). Basic fibroblast growth factor supports undifferentiated human embryonic stem cell growth without conditioned medium. Stem cells, 23(3), 315-323.
[3] Xu, C., Inokuma, M. S., Denham, J., Golds, K., Kundu, P., Gold, J. D., & Carpenter, M. K. (2001). Feeder-free growth of undifferentiated human embryonic stem cells. Nature biotechnology, 19(10), 971.
[4] Grothe, C., & Nikkhah, G. (2001). The role of basic fibroblast growth factor in peripheral nerve regeneration. Anatomy and embryology, 204(3), 171-177.
[5] Takehara, N., Tsutsumi, Y, Tateishi, K., Ogata, T., Tanaka, H., Ueyama, T., ... & Yamagishi, M. (2008). Controlled delivery of basic fibroblast growth factor promotes human cardiosphere-derived cell engraftment to enhance cardiac repair for chronic myocardial infarction. Journal of the American College of Cardiology, 52(23), 1858-1865.
[6] Bennett, S. P., Griffiths, G. D., Schor, A. M., Leese, G. P., & Schor, S. L. (2003). Growth factors in the treatment of diabetic foot ulcers. British Journal of Surgery, 90(2), 133-146.
[7] Mansbridge, J. N., Liu, K., Pinney, R. E., Patch, R., Ratcliffe, A., & Naughton, G. K. (1999). Growth factors secreted by fibroblasts: role in healing diabetic foot ulcers. Diabetes, Obesity and Metabolism, 1(5), 265-279.
[8] Tabata, Y, Yamada, K., Miyamoto, S., Nagata, I., Kikuchi, H., Aoyama, I., ... & Ikada, Y. (1998). Bone regeneration by basic fibroblast growth factor complexed with biodegradable hydrogels. Biomaterials, 19(7-9), 807-815.
[9] MyBioSource (2019). Retrieved from https://www.mybiosource.com/bfgf-recombinant-protein/basic-fibroblast-growth-factor/553055
[10] ThermoFisher (2019). Retrieved from https://www.thermofisher.com/order/catalog/product/13256029
[11] Taguchi S, Ooi T, Mizuno K, Matsusaki H (2015) Advances and needs for endotoxin-free production strains. Appl Microbiol Biotechnol 99:9349-9360
[12] Chen JQ, Fu G, Gai YM, Zheng P, Dw Z, Wen JP (2015) Combinatorial Sec pathway analysis for improved heterologous protein secretion in Bacillus subtilis: identification of bottlenecks by systematic gene overexpression. Microb Cell Factories 14:92
[13] Nguyen TT, Quyen TD, Le HT (2013) Cloning and enhancing production of a detergent-and organic-solvent-resistant nattokinase from Bacillus subtilis VTCC-DVN-12-01 by using an eight-protease-gene-defcient Bacillus subtilisWB800. Microb cell Fact 12: 79
[14] Brückner, R. (1992). A series of shuttle vectors for Bacillus subtilis and Escherichia coli. Gene, 122(1), 187-192.
[15] He, Q., Fu, A. Y, & Li, T. J. (2015). Expression and one-step purification of the antimicrobial peptide cathelicidin-BF using the intein system in Bacillus subtilis. Journal of industrial microbiology & biotechnology, 42(4), 647-653.

What is claim is:

1. A nucleic acid construct comprising an insert, wherein the insert comprises, from the 5′ end to the 3′ end, a polynucleotide sequence that encodes a short peptide affinity tag, a trans-splicing intein derived from Anabaena and an exogenous polypeptide; and wherein the short peptide affinity tag serves as an N-terminal extein of the trans-splicing intein, and the exogenous polypeptide serves as a C-terminal extein of the trans-splicing intein.

2. The nucleic acid construct of claim 1, wherein the intein is an intein of Anabaena DNA polymerase III unit (Asp DnaE).

3. The nucleic acid construct of claim 1 or 2, wherein the intein comprises an amino acid sequence having at least 75% sequence identity to SEQ ID NO:2.

4. The nucleic acid construct of claim 1 or 2, wherein the intein consists of the sequence of SEQ ID NO:2.

5. The nucleic acid construct of any one of claims 1 to 4, wherein the exogenous polypeptide is a fibroblast growth factor (FGF), such as basic fibroblast growth factor (bFGF), especially human bFGF.

6. The nucleic acid construct of any one of claims 1 to 5, wherein the short peptide affinity tag has a length of about 4-15 amino acids, for example, a 5-15 x His tag, especially a 6 x His tag.

7. The nucleic acid construct of any one of claims 1 to 6, wherein the nucleic acid construct further comprises one or more of the following elements: a promoter, an operator, an enhancer, and a ribosome binding site.

8. The nucleic acid construct of any one of claims 1 to 7, wherein from the 5′ end to the 3′ end, a nucleotide sequence that encodes T7 promoter-lac operator-ribosome binding site (RBS)-6 x His tag-Asp DnaE intein-bFGF-T7 transcription terminator.

9. The nucleic acid construct of any one of claims 1 to 8, further comprising a first cloning site upstream of the insert and a second cloning site downstream of the insert, wherein the first cloning site and the second cloning site allow the nucleic acid construct to be inserted into an expression vector.

10. An expression vector comprising the nucleic acid construct of any one of claim 1 to 9.

11. A transformed Bacillus subtilis comprising the expression vector of claim 10.

12. A method of producing an exogenous polypeptide comprising culturing a transformed Bacillus subtilis of claim 11 under conditions that allow the expression of the exogenous polypeptide.

13. The method of producing an exogenous polypeptide of claim 12, further comprising isolating the cultured Bacillus subtilis and then lysing to obtain a cell lysate, followed by isolating the exogenous polypeptide from the cell lysate by sequential use of cation exchange chromatography and heparin-agarose (HA) chromatography.
